(19) United States
(12) Patent Application Publication
(10) Pub. No.: US 2007/0254291 A1
(43) Pub. Date: Nov. 1, 2007

(54) Gene Targeting in Eukaryotic Cells by Group II Intron Ribonucleoprotein Particles

(76) Inventors: Xiaoxia Cui, Saint Louis, MO (US); Alan Lamhowitz, Austin, TX (US); Jamie L. Vernon, Austin, TX (US); Kazuo Watanabe, Funabashi (JP)

Correspondence Address: CALFEE HALTER & GRISWOLD, LLP, 800 SUPERIOR AVENUE, SUITE 1400, CLEVELAND, OH, 44114, US

(21) Appl. No.: 11/629,440

(22) PCT Filed: Jun. 14, 2005
(86) PCT No.: PCT/US05/20894

§ 371 Date: Jun. 27, 2007

Related U.S. Application Data
(60) Provisional application No. 60/579,326, filed on Jun. 14, 2004.

(51) Int. Cl. C12Q 1/68; C12N 15/12

(52) U.S. Cl. 435/6; 435/375; 435/455

(57) ABSTRACT

Provided herein are methods of disrupting DNA substrates in eukaryotic cells and methods of introducing exogenous polynucleotides into target sites in the DNA substrates In certain embodiments the methods comprise introducing a purified group II intron ribonucleoprotein (RNP) particle into the host cell. In certain embodiments the method comprises introducing a group II intron RNP particle and a DNA construct comprising an exogenous polynucleotide flanked by sequences that are homologous to sequences that flank the target site in the endogenous DNA substrate. In certain embodiments, the methods also involve introducing magnesium ions into the eukaryotic cells.

### CROSS REFERENCE TO RELATED APPLICATIONS

This application claims priority to U.S. Provisional Application No. 60/579,326 filed Jun. 14, 2004, which is incorporated herein by reference in its entirety.

### STATEMENT RE GOVERNMENT FUNDING

This work was supported, at least in part, by grant number GM37949 from the Department of Health and Human Services, National Institutes of Health. The United States government has certain rights in this invention.

### FIELD OF THE INVENTION

The present invention relates to methods of disrupting DNA substrates in eukaryotic cells and methods of introducing exogenous polynucleotides into target sites in the DNA substrates, processes which are referred to hereinafter as gene targeting. Such methods employ a purified group II intron ribonucleoprotein (RNP) particle, which comprises an excised group II intron RNA and a group II intron-encoded protein.

### BACKGROUND OF THE INVENTION

Investigators have found it difficult to manipulate complex genomes, such as those found in eukaryotic cells. The methods that have previously been used to disrupt genes or to introduce new sequences into specific loci in cellular genomic DNA (e.g. homologous recombination) and thereby to produce knockout animals have proven to be inefficient and labor intensive. Retroposable elements such as retroviral and lentiviral vectors have been employed for many gene therapy applications. Unfortunately, such gene transfer vectors integrate genes into many different DNA sequences, and unintended integration of the vector near a growth-promoting gene can engender pathological effects. Antisense RNA and RNA interference by ds RNA are unstable and may produce only transient and/or incomplete disruption of a gene's function, which is not inheritable to the next generation. Accordingly, new methods of disrupting specific loci in a DNA substrate, particularly cellular genomic DNA, in eukaryotic cells and of introducing new nucleic acids into specific regions in endogenous DNA substrates are desirable.

### SUMMARY OF THE INVENTION

The present invention provides methods for targeting DNA substrates in eukaryotic cells, particularly animal cells. In one embodiment, the methods are for forming a break at a target site in a single-stranded or double-stranded DNA substrate in the eukaryotic cells. In another embodiment, the methods of the present invention are for forming a break in the endogenous DNA substrate at a target site and incorporating an exogenous polynucleotide into the target site. In another embodiment, the methods are for targeting an exogenous polynucleotide to a predetermined endogenous DNA target sequence in a eukaryotic cell by homologous pairing, particularly for altering an endogenous DNA sequence, such as a chromosomal DNA sequence, typically by targeted homologous recombination. The methods of the present invention employ a group II intron ribonucleoprotein (RNP) particle that is configured to disrupt the DNA substrate at a target site. In certain embodiments, the group II intron RNP particle is a purified particle. The group II intron RNP particle comprises a wild type or modified excised group II intron RNA associated with a wild-type or modified group II intron encoded protein. The group II intron RNA comprises sequences that hybridize to sequences upstream and downstream of the targeted cleavage site.

In one embodiment, the method comprises introducing a group II intron ribonucleoprotein (RNP) particle, preferably a modified group II intron RNP particle, into the eukaryotic cell, and maintaining the eukaryotic cell under conditions that permit the RNP particle to catalyze cleavage of an intracellular DNA substrate at the target site.

In another embodiment, the method comprises introducing the group II intron RNP particle, preferably a modified group II intron RNP particle, into the eukaryotic cell, and maintaining the eukaryotic cell under conditions that permit the group II intron RNP particle to catalyze cleavage of an endogenous DNA substrate at the target site and to incorporate the group II intron into the DNA substrate at the target site. In certain embodiments, the modified group II intron comprises an exogenous polynucleotide encoding a desired protein, peptide or RNA molecule. In certain embodiments, an exogenous polynucleotide encoding a desired product is located in domain IV of the modified group II intron RNA.

In another embodiment, the method comprises introducing into the eukaryotic cell a group II intron ribonucleoprotein (RNP) particle, preferably a modified group II intron RNP particle, configured to introduce single-stranded or, preferably, a double stranded break at a target site in an endogenous DNA substrate in the cell and a DNA construct comprising an exogenous polynucleotide flanked by sequences that are homologous to sequences that flank the target site in the endogenous DNA substrate, and maintaining the eukaryotic cell under conditions that permit the modified RNP particle to introduce the single-stranded or double-stranded break at the target site in the endogenous DNA substrate and that permit insertion of the DNA construct into the target site. Insertion occurs by homologous recombination between the endogenous DNA substrate and the DNA construct. In one embodiment, the RNP particles are introduced into the cells by electroporation or microinjection. In another embodiment, the RNP particles are introduced into the cell by expressing one or more polynucleotides that encode the group II intron RNA and the group II intron encoded protein and that have been introduced into the cell.

In certain embodiments, magnesium ions (Mg²⁺) are also introduced into the cell in order to increase the content of magnesium in the nucleus of the cell. In one embodiment, the intracellular magnesium content of the cell may be increased by introducing a solution comprising 100 mM or greater magnesium into the cell before, after or concurrently with introduction of the RNP particle into the cell or cells. In certain embodiments, the magnesium content of the targeted eukaryotic cell is increased by injecting a solution comprising 100 mM to 1 M of a magnesium salt, preferably MgCl₂, into the cell.

Because the RNP particles can be designed to target specific sequences in the DNA substrate, the methods of the present invention are useful for rendering specific DNA substrates in eukaryotic cells nonfunctional. Thus, the present methods can be used to disrupt specific regions of interest in the genome of a eukaryotic cell. The present methods are also useful for inserting an exogenous polynucleotide into the cleavage site of a target cellular DNA substrate, including targeted regions in the cell's genomic DNA, thus changing the characteristics of the targeted cellular DNA, as well as the RNA and protein molecules encoded by the targeted cellular DNA. It is believed that direct DNA disruption by the methods of the present invention will provide complete and sustained, genetically stable disruption of the DNA of interest.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE FIGURES

FIG. 1 is a diagram showing the interaction between the EBS sequences of a group II intron RNA of the second intron of the S. cerevisiae mitochondrial COX1 gene, hereinafter referred to as the “aI2 intron” RNA and the IBS sequences of a DNA substrate.

FIG. 2 is a diagram depicting the nucleotide sequence of the aI2 intron RNA, SEQ.ID.NO. 1 and the nucleotide sequence of the group II intron RNA of the first intron of the S. cerevisiae mitochondrial COX1 gene, hereinafter referred to as the “aI1 intron” RNA, SEQ.ID.NO.2. Markings above the sequence identify the position of the EBSI sequence and the EBS2 sequence of the wild-type aIl intron RNA and the wild-type aI2 intron RNA.

FIG. 3 shows the L1.LtrB intron DNA sequence and portions of the nucleotide sequence of the flanking exons ltrBE1 and ltrBE2, SEQ.ID.NO.5, and the nucleotide sequence of the open reading frame, of the L1.LtrB intron SEQ. ID. NO. 6.

FIG. 4 depicts a plasmid that was introduced into E. coli for producing purified RNP particles that can be used in the present methods.

FIG. 5 shows the group II intron RNA splicing mechanism and secondary structure. A. Splicing occurs via two sequential transesterification reactions. In the first, nucleophilic attack at the 5′-splice site by the 2′ OH of a bulged A-residue in domain VI results in cleavage of the 5 ′-splice site coupled to formation of lariat intermediate. In the second, nucleophilic attack at the 3′-splice site by the 3′ OH of the cleaved 5′ exon results in exon ligation and release of the intron lariat. B. The conserved secondary structure consists of six double-helical domains (DI-DVI) emanating from a central wheel, with subdomains indicated by lower case letters (e.g., DIVa). The ORF is encoded within DIV (dotted loop), and DIVa is the high-affinity binding site for the intron encoded protein (IEP). Greek letters indicate sequences involved in tertiary interactions. EBS and IBS refer to exon- and intron-binding sites, respectively. As used herein, the term “EBS” also refers to hybridizing sequences in the intron RNA that base pair with sequences in a DNA substrate in the eukaryotic cell. Some key differences between subgroup IIA, IIB, and IIC introns are indicated within dashed boxes, but additional smaller differences are not shown.

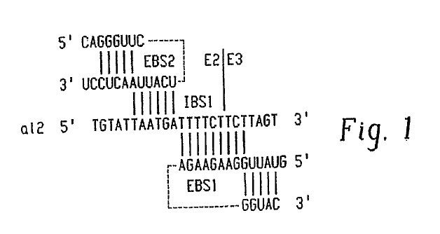

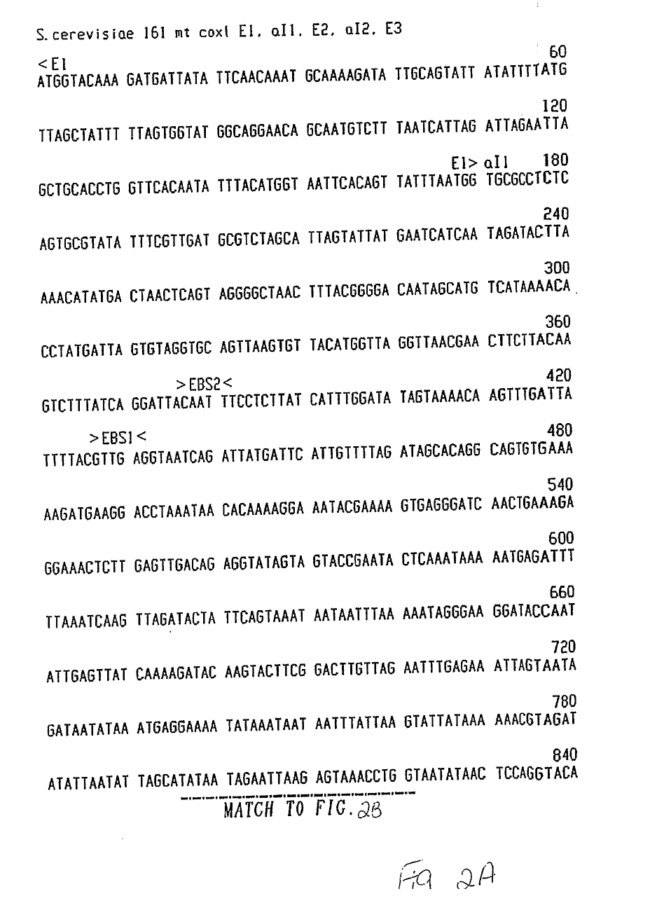

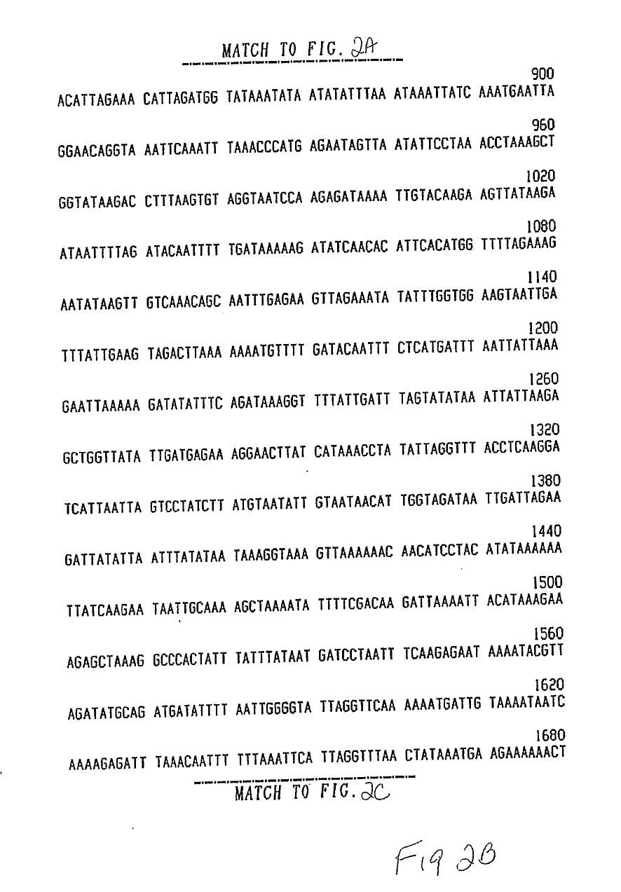

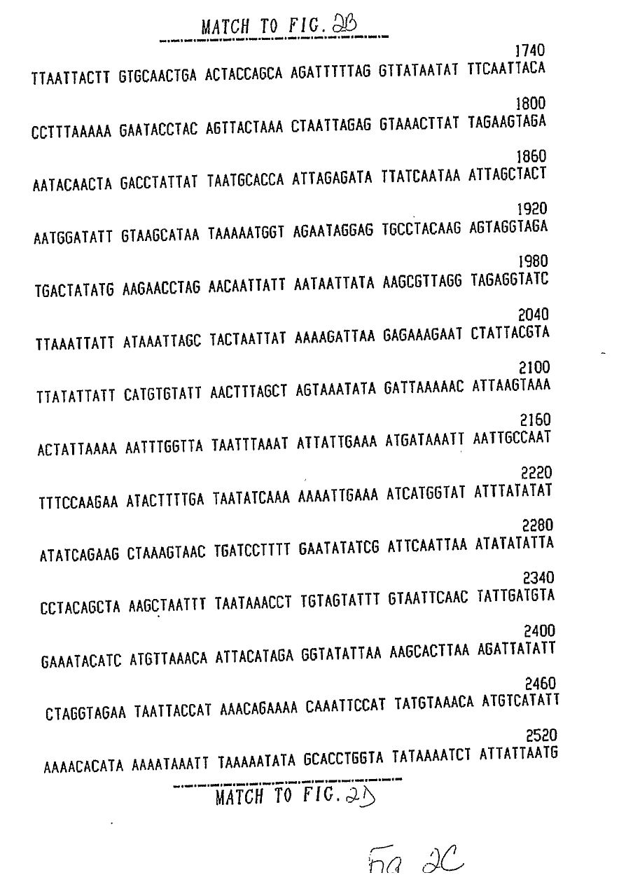

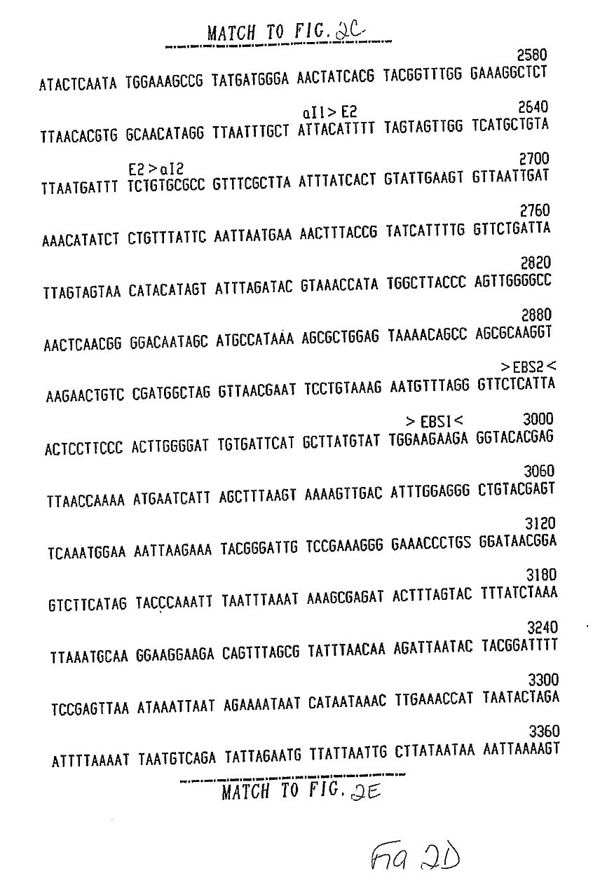

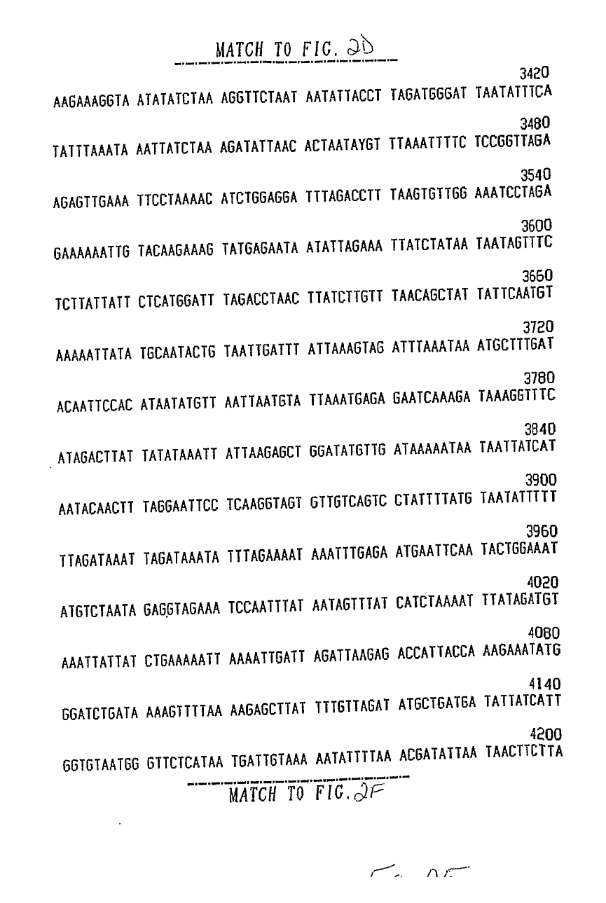

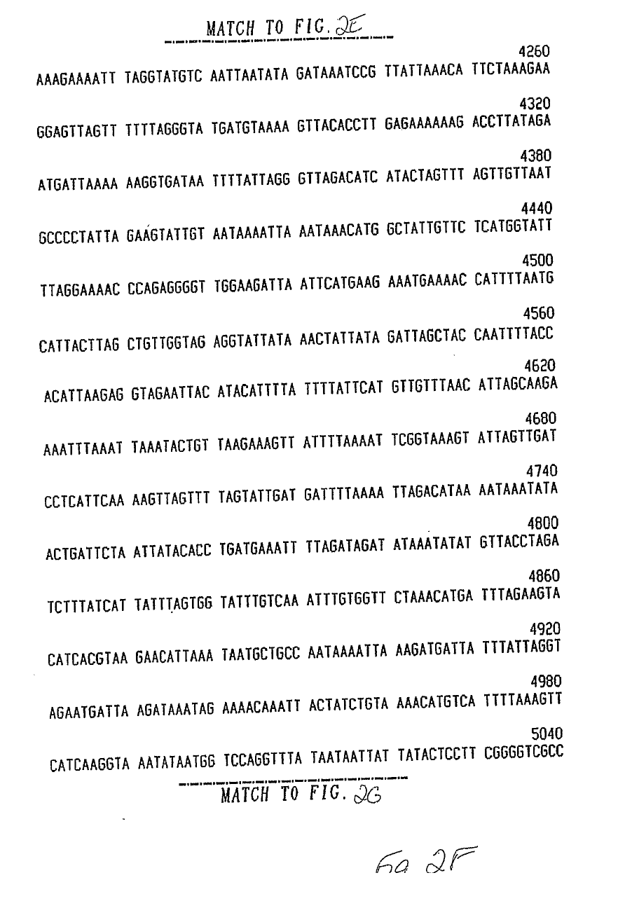

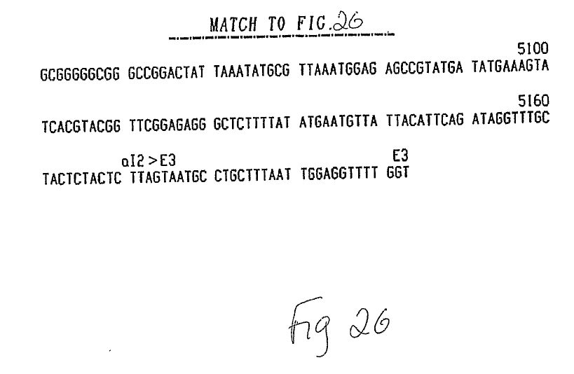

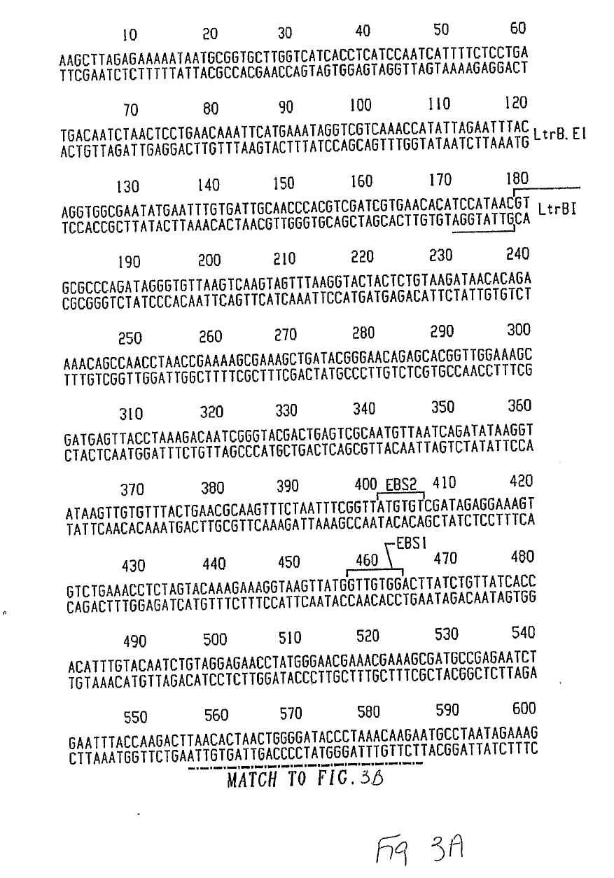

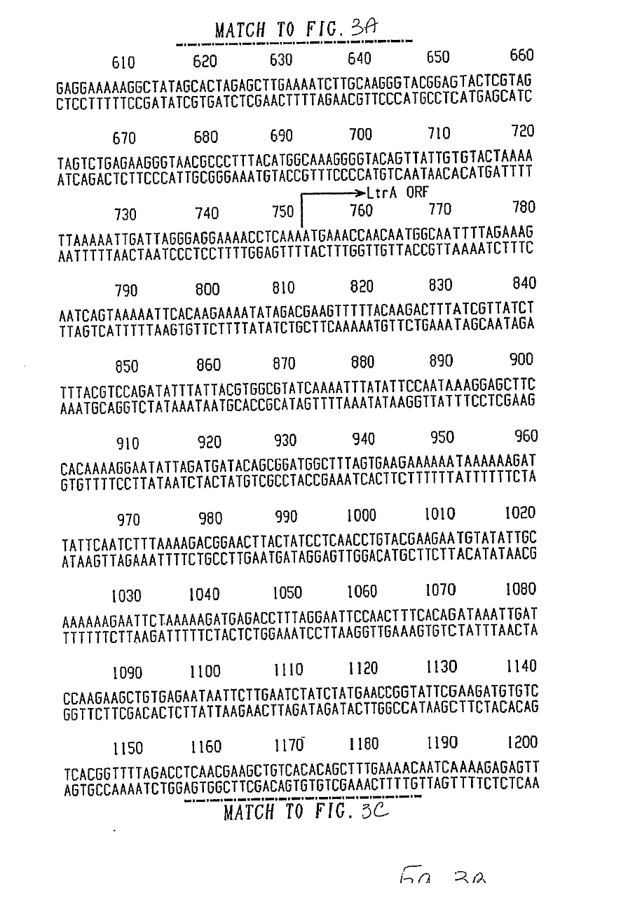

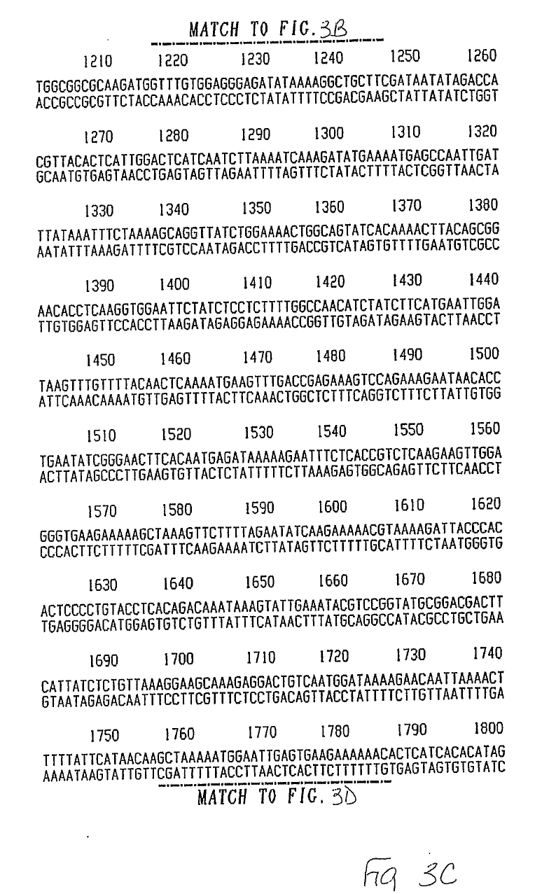

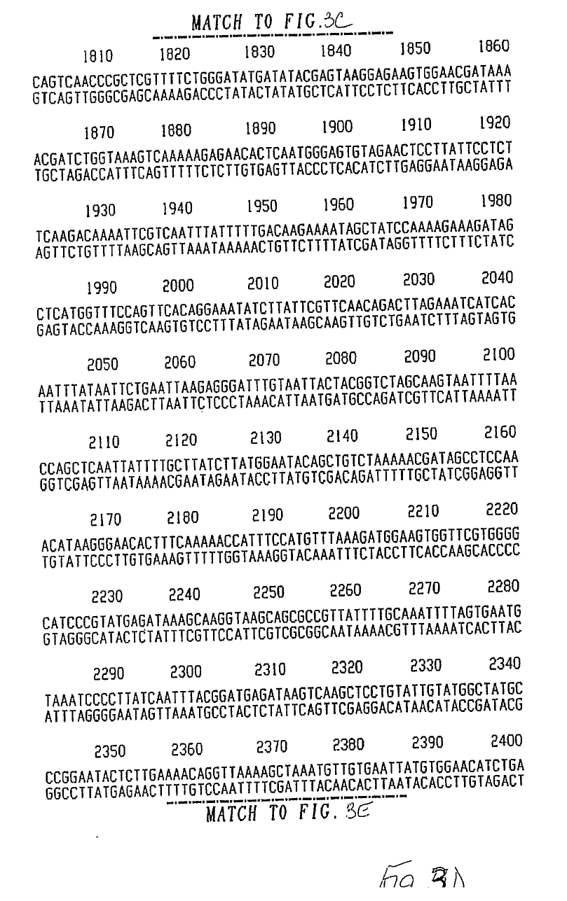

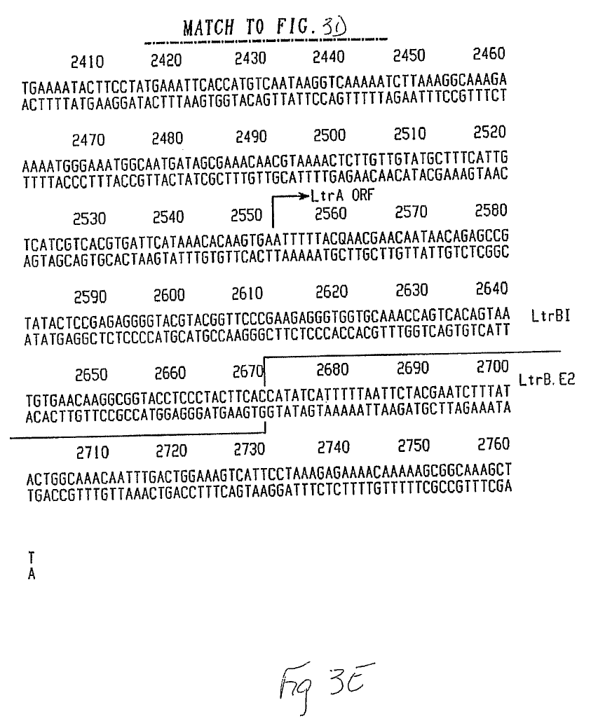

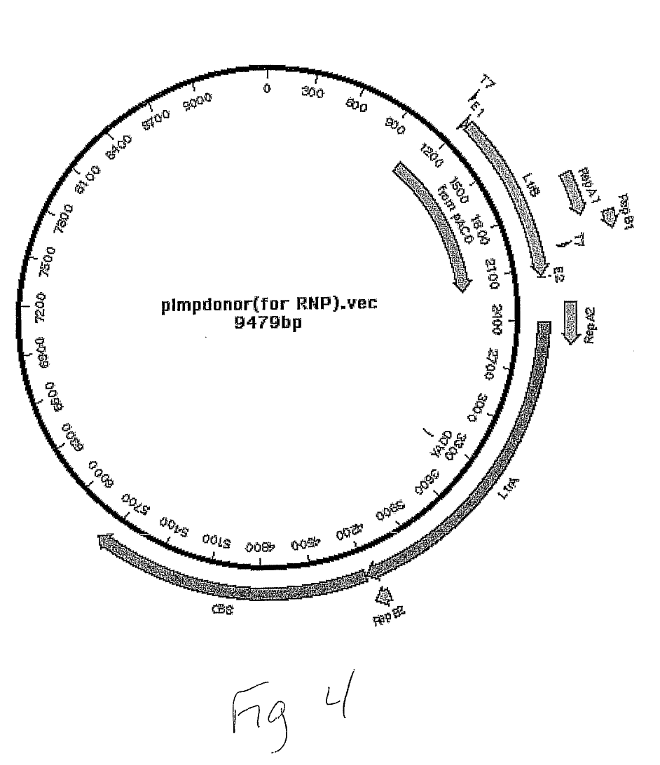

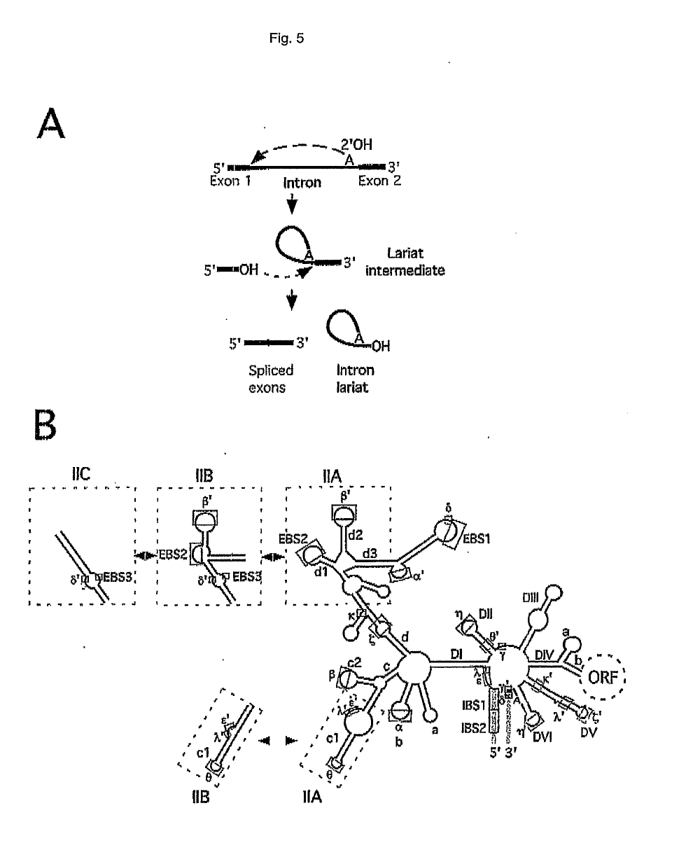

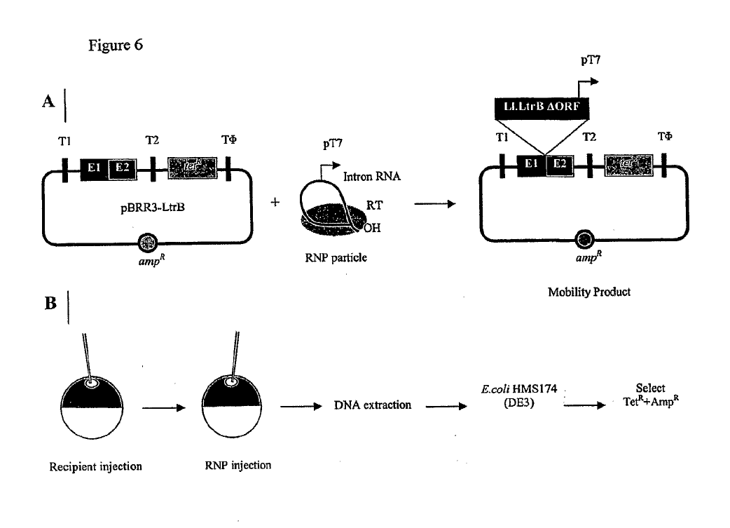

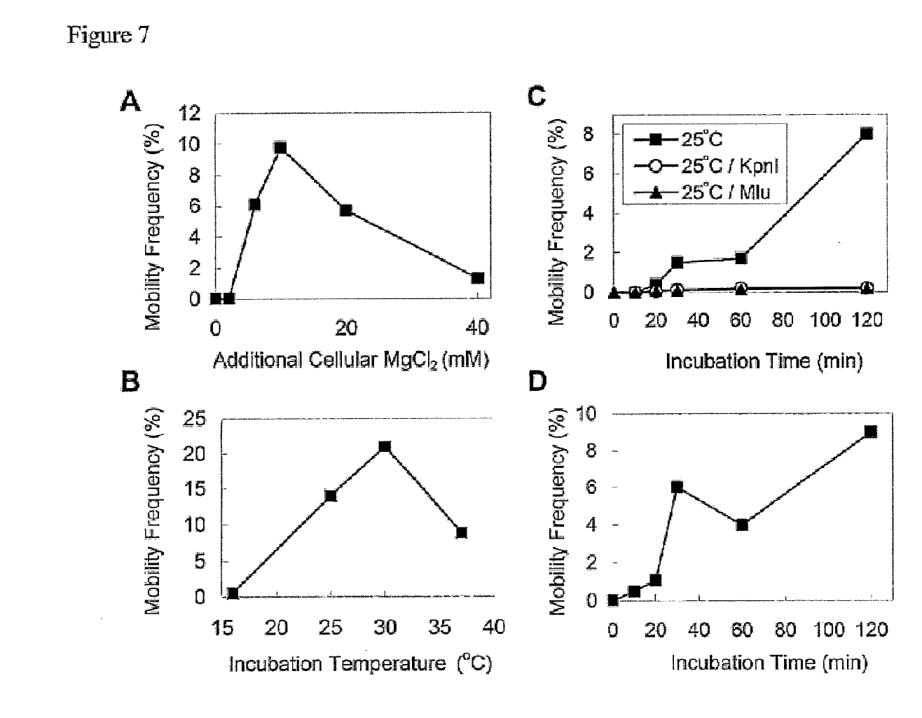

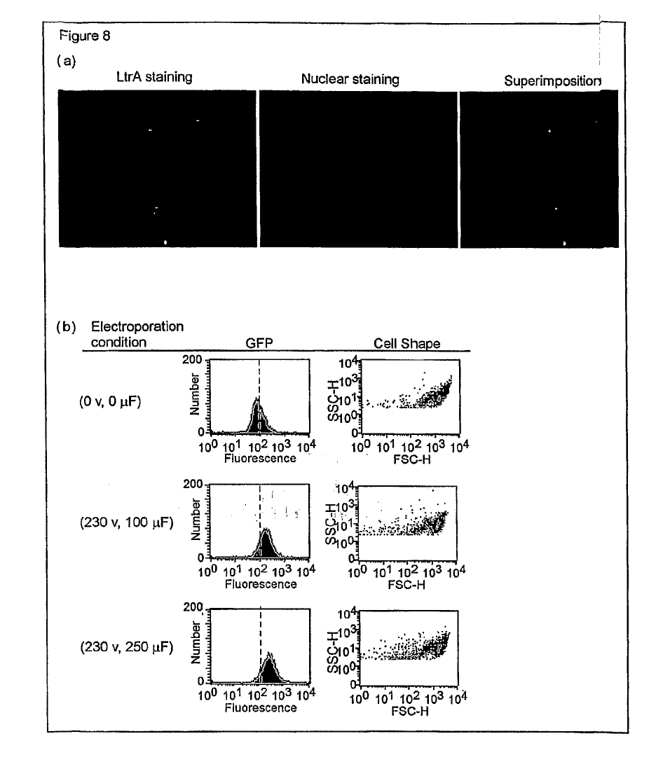

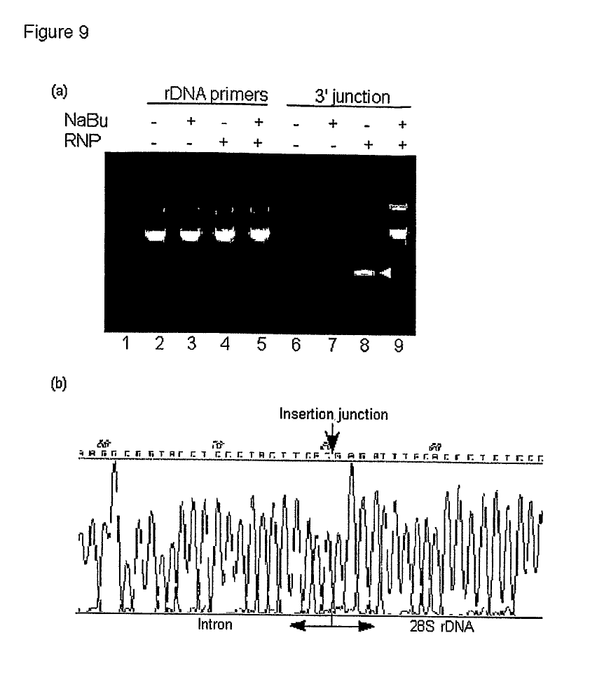

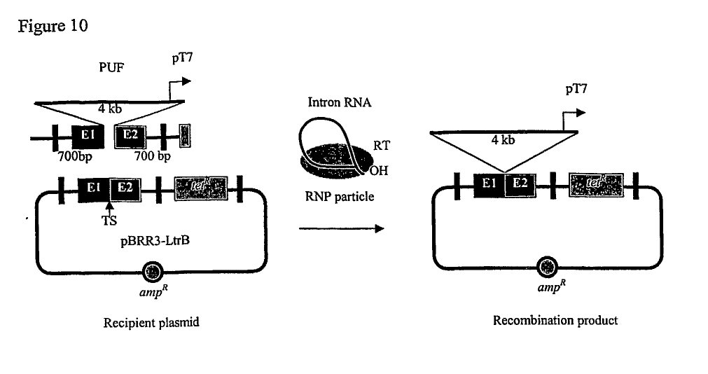

Key to the operation of group II introns are three short sequence elements, referred to hereinafter as “hybridizing regions” that base pair with flanking 5′- and 3′-exon sequences to help position the splice junctions at the intron's active site for both RNA splicing and reverse splicing reactions (FIG. 5B). The sequence elements EBS1 and EBS2 (exon-binding sites 1 and 2) in DI each form 5 to 6 base pairs with the 5′-exon sequences IBS1 and IBS2 (intron-binding sites 1 and 2). As used herein, “IBS” refers to sequences in the target DNA substrate that lie immediately upstream of the targeted cleavage site. In group IIA introns, the sequence δ adjacent to EBS1 base pairs with δ′, typically the first 1-3 nucleotides of the 3′ exon, i.e., the first 1-3 nucleotides downstream of the targeted cleavage site, while in group IIB introns, the 3′ exon base pairs instead with EBS3, located in a different part of DI (FIG. 5B).

FIG. 6. Group II intron integration assay in Xenopus oocyte nuclei. (A) Plasmid targeting assay. The recipient plasmid pBRR3-ltrB contains the L1.LtrB homing site (ligated exon 1 and 2, E1 and E2) cloned upstream of a promoterless tet^{R} gene, and the RNPs contain a 0.9-kb L1.LtrB-ΔORF intron with a phage T7 promoter near its 3′ end. Integration of the L1.LtrB intron into the target site places the intron-borne T7 promoter in front of the tet^{R} gene, allowing it's expression. T1 and T2 are E. coli rrnB transcription terminators, and Tφ is a phage T7 transcription terminator. (B) Protocol for Xenopus microinjection. Recipient plasmids and RNPs were injected separately into oocyte nuclei using different needles. The injected oocytes were incubated at specified times and temperatures, and nucleic acids were isolated and electroporated into E. coli HMS174(DE3). Cells was plated at different dilutions onto LB medium containing tetracycline and tetracycline plus ampicillin, and the frequency of targeting events was calculated as the ratio of Tet^{R}+Amp^{R}/Amp^{R} colonies.

FIG. 7. Parameters for group II intron targeting reactions in Xenopus oocyte nuclei. (A) Mg²⁺-concentration dependence. MgCl₂ was added to recipient plasmid DNA at concentrations ranging from 0 to 2 M, and 20 nl was injected into Xenopus oocyte nuclei, prior to injecting RNPs. The x-axis shows the calculated increase in intracellular Mg²⁺ resulting from the injection, assuming an average oocyte volume of 1 μl. The optimal additional Mg²⁺ concentration (10 mM) corresponds to injection of recipient plasmid DNA in 500 mM MgCl₂. The additional MgCl₂ could also be injected separately with similar results (not shown). (B) Temperature dependence at optimal MgCl₂ concentration. After injecting recipient plasmid DNA and RNPs, oocytes were incubated at the indicated temperature for 120 min prior to isolating nucleic acids. (C and D) Time courses at optimal MgCl₂ concentration. After injecting recipient plasmid DNA and RNPs, oocytes were incubated at 25° (C) and 30° C. (D) for the indicated times prior to isolation of nucleic acids. Nucleic acids isolated from 25° C.-grown cells were untreated or digested with KpnI or MluI prior to transforming E. coli. Each of the experiments was repeated at least twice with essentially the same results.

FIG. 8. Purified RNP can be delivered into cultured cells by electroporation. An RNP containing LtrA with an N-terminal GFP fusion was used to test electroporation conditions. (a) Fluorescence microscopy detects GFP in the cells, although not necessarily in the nucleus. (b) Flow cytometry shows uptake of GFP-containing RNP particles into the cells, demonstrated by the upshift of fluorescence intensity in the transfected cells.

FIG. 9. Successful chromosomal targeting in tissue culture by electroporation of RNPs. (a) Nested PCRs with two sets of primers: rDNA primers and 3′ insertion junction primers, consisting one intron primer and one rDNA primer. Template DNA was purified from cells electroporated with or without RNP and treated with or without NaBu. (b) Sequencing of the PCR band in lane 8 resulted correct intron/rDNA junction

FIG. 10. Schematic of assay of group II intron-RNP stimulated homologous recombination.

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

I. Definitions

“Group II intron DNA,” as used herein, is a specific type of DNA present in bacteria and in organelles, particularly the mitochondria of fungi, yeast and plants and the chloroplast of plants. The group II intron RNA molecules, that is, the RNA molecules encoded by the group II introns, share similar secondary and tertiary structures. The group II intron RNA molecules typically have six domains. Domain IV of the group II intron RNA contains the nucleotide sequence which encodes the “group II intron-encoded protein.”

“Excised group II intron RNA,” as used herein, refers to an RNA that is a transcript of the group II intron DNA that lacks flanking exon sequences.

Group II intron encoded protein,” as used herein, is a protein encoded by an open reading frame within a group II intron. The group II intron-encoded protein comprises an X domain and a reverse transcriptase domain. The X domain of the protein is associated with maturase activity. In some cases, the proteins also comprise an En domain having a DNA endonuclease motif. As used herein, group II intron-encoded proteins also encompass modified group II intron-encoded proteins that have additional or altered amino acids at the N terminus, or C terminus, or alterations in the internal regions of the protein, as well as wild-type group II intron-encoded proteins.

“Modified,” as used herein, refers to DNA, RNA or proteins which differ from the wild-type form of the DNA, RNA, or protein. In the case of DNA or RNA, modified refers to one or more of substitutions, additions or deletions of nucleotides in the DNA or RNA sequence, such that the modified sequence is different from the normal, wild-type sequence. Modified can refer to substitutions, additions or deletions of nucleotides in a sequence within DNA or RNA that does not encode a protein, for example, one or more of exon binding site (EBS)1, exon binding site 2 (EBS2) and δ regions of the group II intron. Modified can also refer to substitutions, additions or deletions of nucleotides, as compared to normal wild type, within a protein-encoding sequence of the DNA or RNA. The protein encoded by such a modified protein-encoding DNA or RNA sequence could itself be modified in that it could have one or more of substitutions, additions or deletions of amino acids within its protein sequence as compared to the normal, wild-type sequence of the protein.

“DNA recognition sites,” as used herein, refer to the sequence of nucleotide bases within the DNA substrate which are recognized by the RNP particle, or components thereof, as signals to cleave the DNA substrate and then insert nucleic acid molecules into the substrate. DNA recognition sites can also be referred to as “targets” since these are sites into which nucleic acid molecules are inserted.

“DNA substrate,” as used herein, means the DNA molecule containing DNA recognition sites which are cleaved by the group II intron RNP particles of the present invention and into which an exogenous polynucleotide may be inserted.

“Promoter,” as used herein, refers to sequences in DNA which mediate initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements downstream of the transcription initiation site. The individual sequence elements function as sites on the DNA where RNA polymerases, and transcription factors that facilitate positioning of RNA polymerases on the DNA, bind.

“Flanking DNA”, as used herein, refers to a segment of DNA that is collinear with and adjacent to a particular point of reference.

“Homologous”, as used herein, means two or more nucleic acid sequences that are either identical or similar enough that they are able to hybridize to each other or undergo intermolecular exchange.

As used herein, the terms “endogenous DNA sequence” and “target sequence” refer to polynucleotide sequences contained in a eukaryotic cell. Such sequences include, for example, chromosomal sequences (e.g., structural genes, promoters, enhancers, recombinatorial hotspots, repeat sequences, integrated proviral sequences), episomal sequences (e.g., replicable plasmids or viral replication intermediates) In some embodiments, the endogenous DNA target sequence will be other than a naturally occurring germline DNA sequence (e.g., a transgene, parasitic, or mycoplasmal or viral sequence).

An “exogenous polynucleotide”, as used herein, is a polynucleotide which is transferred into a eukaryotic cell but which has not been replicated in that host cell. The exogenous polynucleotide can be an entire gene encoding an entire desired product or a gene portion which encodes, for example, the active or functional portion(s) of the product. The product can be, for example, a hormone, a cytokine, an antigen, an antibody, an enzyme, a clotting factor, a transport protein, a receptor, a regulatory protein, a structural protein, an anti-sense RNA, a ribozyme or a protein or a nucleic acid which does not occur in nature (i.e., a novel protein or novel nucleic-acid). The DNA can be obtained from a source in which it occurs in nature or can be produced, using genetic engineering techniques or synthetic processes. The exogenous polynucleotide can encode one or more therapeutic products.

The present invention provides methods for altering specific sites or sequences in DNA substrates in eukaryotic cells. In certain embodiments, the method comprises injecting or electroporating purified wild-type or modified group II intron derived RNP particles into the cells. The purified RNP particles that are injected or electroporated into eukaryotic cells in accordance with the present methods are enzymes that are capable of cleaving double-stranded DNA substrates at specific recognition sites. In certain embodiments, injection or electroporation of the purified RNP particles into the eukaryotic cell can result in incorporation of the wild-type or modified group II intron into the DNA substrate at the cleavage site. Reaction of the targeted DNA substrate with the group II intron RNP particle in cells results initially in the insertion of the group II intron RNA molecule of the RNP particle into one strand of the double-stranded DNA substrate at the cleavage site, then synthesis of a cDNA molecule which is complementary to the group II intron RNA molecule into the other strand of the double-stranded DNA substrate. Formation of the heteroduplex in the cleavage site occurs by a mechanism in which the excised group II intron RNA reverse splices directly into the DNA target site and is then reverse transcribed by the intron-encoded protein. Over time, this heteroduplex structure is converted to a double stranded DNA structure that encodes the group II intron.

The purified RNP particles used in the present gene targeting methods are derived from group II introns. Wild-type group II introns are found in bacteria and organellar genomes, primarily mitochondria and chlioroplast of lower eukaryotes and higher plants. They are also found in both gram-positive and gram negative bacteria, and a few archaea. Particularly good results have been achieved using RNP particles derived from bacterial group II introns.

The present application contemplates methods which employ purified RNP particles comprising sequences that encode wild-type and modified group II intron RNA and wild-type and modified group II intron encoded proteins derived from group IIA, group IIB, and group IIC introns.

The group II intron RNP particles used in the present methods comprise a group II intron-encoded protein which is bound to an excised group II intron RNA whose sequence is identical to a group II intron RNA that is found in nature, i.e., a wild-type group II intron RNA, or an excised group II RNA whose sequence is different from a group II intron RNA that is found in nature, i.e., a modified, excised group II intron RNA molecule. Modified excised group II intron RNA molecules, include, for example, group II intron RNA molecules that have nucleotide base changes or additional nucleotides in the internal loop regions of the group II intron RNA, preferably the internal loop region of domain IV, and group II intron RNA molecules that have nucleotide base changes in the hybridizing regions of domain I (e.g. EBS1, EBS2, or 8). RNP particles in which the group II intron RNA has nucleotide base changes in the hybridizing region, as compared to the wild type, typically have altered specificity for the DNA substrate of the wild-type RNP particle.

Targeting of the RNP particles to specific regions of the DNA substrate involves base pairing of the excised, modified or wild-type group II intron RNA of the RNP particle to a specific region of the targeted DNA substrate. The group II intron RNA has two sequences, EBS1 and EBS2, that are capable of hybridizing with two intron RNA-binding sequences, IBS1 and IBS2, on one strand of the DNA substrate, hereinafter referred to as the “top” strand for convenience. Additional interactions occur between the intron-encoded protein and regions in the DNA substrate flanking the IBS1 and IBS2 sites. As denoted herein, nucleotides that are located upstream of the cleavage site have a (−) position relative to the cleavage site, and nucleotides that are located downstream of the cleavage site have a (+) position relative to the cleavage site. Thus, the cleavage site is located between nucleotides −1 and +1 on the top strand of the double-stranded DNA substrate. The IBS1 sequence and the IBS2 sequence lie in a region of the DNA substrate which extends from about position −1 to about position −14 relative to the cleavage site. Group IIA intron RNA molecules also comprise a sequence referred to as delta (δ) that base pairs with the nucleotides in the 3′ exon, typically +1 to +3 of the DNA substrate, a sequence that is referred to as δ′. Group IIB intron RNA molecules comprise a sequence referred to as EBS3 that base pairs with nucleotide residues in the 3′ exon of the targeted DNA substrate. The δ sequence is located in domain I of the group IIA intron RNA, while the EBS3 sequence is located in a different region of domain I of the group IIB intron RNA. (See FIG. 5)

EBS1 is located in domain I of the group II intron RNA and comprises from about 5 to 7 nucleotides that are capable of hybridizing to the nucleotides of the IBS1 sequence of the substrate. EBS2 is located in domain I of the group II intron RNA upstream of EBS1 and comprises from about 5 to 7 nucleotides that are capable of hybridizing to the nucleotides of IBS2 sequence of the substrate. In order to cleave the substrate efficiently, it is preferred that the δ sequence or the EBS3 sequence of the group II intron RNA, be complementary to the nucleotides in the 3′ exon in the top strand of the substrate.

Examples of group II intron RNP particles which may be used in the present methods include, but are not limited to, the aI2 RNP particle, the all RNP particle, and the L1.LtrB RNP particle. The aI2 RNP particle comprises a wild-type or modified group II intron RNA of the second intron of the S. cerevisiae mitochondrial COX1 gene, hereinafter referred to as the “aI2 intron” RNA, bound to a wild-type or modified aI2 intron encoded-protein. EBSI of the aI2 intron RNA comprises δ nucleotides and is located at position 2985-2990 of the wild-type sequence. EBS1 of the wild-type aI2 intron RNA has the sequence 5′-AGAAGA. EBS2 of the aI2 intron RNA comprises δ nucleotides and is located at positions 2935-2940. EBS2 of the wild-type aI2 intron RNA has the sequence 5′-UCAUUA.

The all RNP particle comprises an excised, wild-type or modified group II intron RNA of the first intron of the S. cerevisiae mitochondrial COX1 gene, hereinafter referred to as the “all intron” RNA, and a wild-type or modified all intron-encoded protein. EBS1 of the all intron RNA comprises 6 to 7 nucleotides and is located at position 426-431. EBS1 of the wild-type all intron RNA has the sequence 5′-CGUUGA. EBS2 of the all intron RNA comprises 5 to 6 nucleotides and is located at positions 376-381. EBS2 of the wild-type all intron RNA has the sequence 5′-ACAAUU.

The L1.LtrB RNP particle comprises an excised, wild-type or modified L1.LtrB group II intron RNA of the Lactococcus lactis ltrB gene, hereinafter referred to as the “L1.LtrB intron” RNA, and a wild-type or modified L1.LtrB intron-encoded protein, hereinafter referred to as the LtrA protein. The sequence of the L1.LtrB intron is shown in the attached figure. The EBS1 of the L1.LtrB intron RNA comprises 7 nucleotides and is located at positions 457 to 463. The EBS1 sequence of the wild-type L1.LtrB intron RNA has the sequence 5′-GUUGUGG. The EBS2 of the L1.LtrB intron RNA comprises 6 nucleotides and is located at positions 401 to and including 406. The EBS2 sequence of the wild-type L1.LtrB intron RNA has the sequence 5′AUGUGU. The intron-encoded protein is a multifunctional reverse transcriptase (RT), which binds to the intron to stabilize the catalytically active RNA structure for both RNA splicing and reverse splicing. In certain embodiments, e.g., the L1.LtrB and related group II introns, the intron-encoded protein also has C-terminal DNA-binding (D) and DNA endonuclease (En) domains. (Lambowitz, A. M. and Zimmerly, S. Mobile group II introns. Amiu. Rev. Genet. 38, 1-35, 2004.

). While not wishing to be bound by theory, it is believed that the group II intron RNPs initiate disruption of the DNA substrate by recognizing DNA target sites with the intron-encoded protein recognizing a small number of positions and triggering local DNA unwinding, enabling the intron RNA to base pair to a 14-16 nt region of the DNA target site (EBS/IBS and δ/δ′). The intron RNA then reverse splices into one strand of the DNA target site, while the intron-encoded protein uses its En domain to cleave the opposite strand and uses the 3′ end at the cleavage site as a primer for reverse transcription of the inserted intron RNA. The resulting intron cDNA is integrated by host cell DNA recombination or repair enzymes (Lambowitz and Zimmerly, 2004.)

The modified RNP particle can catalyze the cleavage of DNA substrates and the insertion of nucleic acid molecules at new recognition sites in the DNA substrate. Because the recognition site of the DNA substrate is recognized, in part, through base pairing with the excised group II intron RNA of the functional RNP particle, it is possible to control the site of nucleic acid insertion within the DNA substrate. This is done by modifying the EBS1 sequence, the EBS2 sequence, the 8 sequence, the EBS3 sequence or combinations thereof. Methods of modifying group II intron RNP particles such that they bind to and catalyze the cleavage of DNA substrates at different recognition sites are described in U.S. Pat. Nos. 5,698,421 and 6,027,895, both of which are incorporated herein by reference.

DNA molecules encoding modified group II intron RNA containing desired EBS sequences which hybridize to corresponding nucleotides on substrate DNA or containing additional nucleotides (e.g. a polynucleotide encoding a drug resistance marker) in domain IV may be prepared using standard genetic engineering procedures, such as in vitro site-directed mutagenesis.

Because the group II intron RNP particles of the present invention recognize their DNA target sites mainly by base pairing of the intron RNA, they can be targeted to insert into different DNA sites simply by modifying the intron RNA. This feature, combined with their very high insertion frequency and specificity, makes it possible to use the functional RNP particles of the present invention as programmable gene-targeting vectors. Additionally, group II introns can be used for the site-specific chromosomal insertion of cargo genes cloned in domain IV of the group II intron RNA and to introduce targeted double-strand breaks, which stimulate homologous recombination with a co-transformed DNA fragment, enabling the introduction of point mutations.

The methods of the present invention can be used to repair defective genes in the cells or to disrupt undesired gene, for example viral genes or oncogenes, contained within the eukaryotic cell. The methods of the present invention can be used to make transgenic animals, for example by injecting the purified RNP particles into fertilized eggs or zygotes. The methods of the present invention can be used to make knockout animals, for example knockout mice, by electroporating the purified RNP particles into embryonic stem (ES) cells or zygotes. The method of the present invention can also be used to prepare knockout libraries, for example by electroporating modified RNP particles having randomized EBS1, ESB2, and 6 or EBS3 sequences into tissue culture cells or mouse ES cells. The methods of the present invention can be used to introduce an exogenous polynucleotide into a DNA substrate in sperm, unfertilized, and fertilized oocytes, and cultured animal or plant cells. All of these uses are contemplated embodiments of the present invention.

Preparation of the Purified RNP Particle

In recent years, methods have been developed for preparing purified group II intron RNP particles whose excised group II RNAs have a wild-type sequence and purified group II intron RNP particles whose excised group II RNAs have a modified sequence. Such methods are described in U.S. Pat. No. 5,804,418 and US application 20030104352. The description of such methods are specifically incorporated herein by reference.

In one embodiment, the purified RNP particle is prepared by co-expressing a wild-type or modified group intron RNA, preferably, the DIV-deletion form of the intron (ΔORF) and a group II intron-encoded protein in E. coli. The intron RNA is flanked by exon sequences, from which it is spliced in E. coli to form a lariat. The group II intron encoded protein preferably is linked to a tag which facilitates isolation of the RNP particle from the transformed E. coli cells. The tag sequences are preferably at the 5′ or 3′ end of the open reading frame sequence of the protein. Suitable tag sequences include, for example, sequences which encode a series of histidine residues, the Herpes simplex glycoprotein D, i.e., the HSV antigen, glutathione S-transferase, or as described below in the examples an chitin-intein tag which allows purification of the RNP particles on a chitin affinity column and removal of the tag by treatment of the purified particles with dithiothreitol. Typically, the DNA molecule also comprises nucleotide sequences that encode a replication origin and a selectable marker. In certain embodiments, a nuclear localization signal (NLS) is linked to group II intron-encoded protein to localize RNPs to the nucleus in eukaryotic cells. (See, copending and commonly assigned PCT Application ______, which is being filed concurrently herewith and claims priority to U.S. Provisional Application No. 60/579,212, filed on Jun. 14, 2004.)

In another embodiment, the group II intron RNP particles are prepared by a procedure involving the following 3 steps: 1) self-splicing of in vitro transcribed intron RNA, 2) reconstitution of the self-spliced RNA and independently purified intron-encoded protein under high salt condition, and 3) ultracentrifugation of the reconstituted mixture. After the ultracentrifugation, pelleted RNPs are resuspended in a buffer and stored at −70° C. Following is a detailed description of the procedure.

To obtain self-spliced intron RNA, in vitro transcribed precursor RNAs (final concentration 0.5 μg/μl) in a medium (final volume 1000μl) containing 25 mM Tris-HCl (pH 7.5), 50 mM MgCl₂, and 1.25 M NH₄Cl, are incubated on warm water at 55° C. in a container (volume about 300-500 ml). The mixture is cooled down naturally to 37° C. by allowing the mixture to stand at room temperature for about 30 minutes. Then the mixture is kept at 37° C. for 3 hr. After the incubation, 2 volumes of ethanol are added and the mixture is stored at −20° C. for 30 min. Then it is centrifuged to obtain ethanol-precipitated RNA. The RNA pellets are washed with 70% ethanol once, and resuspended in 200-500 μl of water. The concentration of the preparation is measured by UV spectrometer (OD₂₆₀) and the samples are stored at −70° C. till next use.

The self-spliced intron RNAs (final concentration 20 nM) and independently purified intron-encoded proteins (final concentration 40 nM) are reconstituted by mixing them in a media (final volume 10 ml) containing, 40 mM Tris-HCl (pH 7.5), 5 mM MgCl₂, and 450 mM NaCl. Tubes containing the mixture are incubated at 30° C. for 1 hr. RNA and protein concentrations can be increased 5 times without interfering efficiency of reconstitution.

The reconstituted RNP complexes are precipitated by ultracentrifugation. Reconstituted mixtures are transferred to ultracentrifuge tubes and spanned at 50,000 rpm at 4° C. with the 70Ti rotor for 17 hr. After removing supernatant completely, transparent pellets are resuspended in buffer (volume about 50 μl) containing 40 mM Tris-HCl (pH 7.5), 10 mM MgCl₂, and 10 mM KCl. Insoluble materials are removed by centrifugation. This final preparation is stored at −70° C. for at least months without losing any activity.

Incorporation of the Purified RNP Particle into Eukaryotic Cells

In certain embodiments, the purified RNP particles are incorporated into cells by electroporation or microinjection. Thus, the present method is particularly useful for modifying DNA substrates in any eukaryotic animal, or plant amenable to introduction of the RNP's by microinjection or electroporation, including xenopus oocytes, zebrafish, Drosophila, C. elegans, rat and mouse zygotes or embryos, and cultured cells. The present targeting methods can also be used to alter targeted endogenous genes in sperm, fertilized and unfertilized eggs, embryonic stem cells, and plant cells.

Incorporation of a DNA Construct into the Target DNA Substrate

In certain embodiments, the methods of the present invention comprise incorporating a group II intron RNP particle and a DNA construct comprising an exogenous polynucleotide into the eukaryotic cell, and maintaining the cell under conditions that permit the group II intron RNP particle to cause formation of a double-stranded break in an endogenous DNA substrate at a target site, and incorporation of the DNA construct into the target site. In certain embodiments, the DNA construct comprises sequences “homologous” to nucleic acid sequences flanking a desired insertion site in the targeted DNA substrate. The flanking homology sequences, referred to as “homology arms”, direct the construct to a specific location within the targeted DNA substrate by virtue of the homology that exists between the homology arms and the corresponding endogenous sequence and introduction of exogenous polynucleotide into the insertion site by a process referred to as “homologous recombination”. Such flanking sequence can be present at one or, preferably, both ends of the exogenous polynucleotide. If two flanking sequences are present, one should be homologous to a first region of the target and the other should be homologous to a second region of the target.

In certain embodiments, the DNA construct comprises an exogenous DNA encoding a desired product, targeting sequences for homologous recombination and, optionally, DNA encoding one or more selectable markers. The total length of the DNA construct will vary according to the number of components (exogenous DNA, targeting sequences, selectable marker gene) and the length of each. The entire construct length will generally be at least 20 nucleotides. In a construct in which the exogenous DNA has sufficient homology with genomic DNA to undergo homologous recombination, the construct may include a single component, the exogenous DNA. In this embodiment, the exogenous DNA, because of its homology, serves also to target integration into genomic DNA and additional targeting sequences are unnecessary. Such a construct is useful to knock out, replace or repair a resident DNA sequence, such as an entire gene, a gene portion, a regulatory element or portion thereof or regions of DNA which, when removed, place regulatory and structural sequences in functional proximity. It is also useful when the exogenous DNA carries a selectable marker.

In another embodiment, the DNA construct includes exogenous DNA and one or more separate targeting sequences, generally located at both ends of the exogenous DNA sequence. Such a construct is useful to integrate exogenous DNA encoding a therapeutic product, such as a hormone, a cytokine, an antigen, an antibody, an enzyme, a clotting factor, a transport protein, a receptor, a regulatory protein, a structural protein, an anti-sense RNA, a ribozyme or a protein or a nucleic acid which does not occur in nature. In particular, exogenous DNA can encode one of the following: Factor VIII, Factor IX, erythropoietin, alpha-1 antitrypsin, calcitonin, glucocerebrosidase, growth hormone, low density lipoprotein (LDL) receptor, IL-2 receptor and its antagonists, insulin, globin, immunoglobulins, catalytic antibodies, the interleukins, insulin-like growth factors, superoxide dismutase, immune responder modifiers, parathyroid hormone, interferons, nerve growth factors, tissue plasminogen activators, and colony stimulating factors. Such a construct is also useful to integrate exogenous DNA which is a therapeutic product, DNA sequences which bind to a cellular regulatory protein, DNA sequences which alter the secondary or tertiary chromosomal structure and DNA sequences which are transcriptional regulatory elements.

In all embodiments of the DNA construct, the exogenous DNA can encode one or more products thus making it possible to deliver multiple products.

Replacement of a Regulatory Sequence of a Gene by Homologous Recombination

As taught herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. (Alternatively, sequences which affect the structure or stability of the RNA or protein produced may be replaced, removed, added, or otherwise modified by targeting, including polyadenylation signals, mRNA stability elements, splice sites, leader sequences for enhancing or modifying transport or secretion properties of the protein, or other sequences which alter or improve the function or stability of protein or RNA molecules).

Several embodiments are possible. First, the targeting event may be a simple insertion of the regulatory sequence, placing the gene under the control of the new regulatory sequence (for example, inserting a new promoter or enhancer or both upstream of a gene). Second, the targeting event may be a simple deletion of a regulatory element, such as the deletion of a tissue-specific negative regulatory element. Third, the targeting event may replace an existing element; for example, a tissue-specific enhancer can be replaced by an enhancer that has broader or different cell-type specificity than the naturally-occurring elements. In this embodiment the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the-property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene, mouse hypoxanthine phosphoribosyl transferase (mHPRT) gene,or the bacterial xanthine-guanine phosphoribosyltransferase (gpt) gene.

Incorporation of the Group II intron RNP particle and DNA Construct into Eukaryotic Cells

The group II intron RNP particle can be introduced into the cell by microinjection or electroporation. Alternatively, the group II intron RNP particle can be introduced into the eukaryotic cell by expression of one or more polynucleotides that encode the modified group II intron RNA and group II intron encoding protein and that have been introduced into the cell using conventional methods.

The DNA construct can be linear or circular and can be introduced into the cell by electroporation, lipid-mediated transfection, Calcium phosphate precipitation, peptide vecter-mediated transfection (Morris, M. C., Depollier, J., Mery, J., Heitz, F., & Divita, G. (2001) A peptide carrier for the delivery of biologically active proteins into mammalian cells. Nat. Biotecluiol. 19, 1173-1176), etc.

The following examples of methods for introducing purified RNP particles into eukaryotic cells and using such particles to cleave targeted DNA substrates in the cells are included for purposes of illustration and are not intended to limit the scope of the invention.

### EXAMPLES

### Example 1

### Modification of DNA Substrates In Xenopus laevis

### Materials and Methods

Recombinant plasmids. pACD2 and pACD3 are intron-donor plasmids used for bacterial gene targeting (Guo, H., Karberg, M., Long, M., Jones, J. P. 3rd, Sullenger, B., Lambowitz, A. M. (2000) Group II introns designed to insert into therapeutically relevant DNA target sites in human cells. Science. 289, 452-457; Karberg, M., Guo, H., Zhong, J., Coon, R., Perutka, J., and Lambowitz, A. M. (2001) Group II introns as controllable gene targeting vectors for genetic manipulation of bacteria. Nat. Biotechnol. 19, 1162-1167). They contain a 0.9-kb L1.LtrB-ΔORF intron and flanking exons cloned downstream of a T7lac promoter in the vector pACYC184, which carries a cap^{R} gene. In both plasmids, the LtrA ORF is cloned just downstream of the 3′ exon; in pACD2, the L1.LtrB-ΔORF intron contains a phage T7 promoter inserted in intron DIV for use in intron mobility assays. The recipient plasmid pBRR3-ltrB contains the L1.LtrB homing site (ligated exon 1 and 2 of the ltrB gene from positions −178 upstream to +90 downstream of the intron-insertion site) cloned upstream of a promoterless tet^{R} gene in an AmP^{R} pBR322-derivative (FIG. 6A; Guo et al. 2000; Karberg et al. 2001). Closed-circular plasmid DNA were purified in a CsCl-ethidium bromide gradient (Sambrook, J., Fritsch, E. F., Maniatis, T. (1989) Molecular cloning: A laboratory manual, 2nd ed. Cold Spring Harbour Laboratory Press, New York) and dissolved into water.

Preparation of Group II Intron RNPs.

A computer algorithm was used to identify target sites in Xenopus laevis genes and design primers for modifying the intron to insert into those sites (Perutka, J., Wang, W., Goerlitz, D., and Lambowitz, A. M. (2004) Use of computer-designed group II introns to disrupt Escherichia coli DexH/D-box protein and DNA helicase genes. J. Mol. Biol. 336, 421-439). Modified L1.LtrB intron RNPs were reconstituted in vitro. The 0.9-kb L1.LtrB-ΔORF intron and flanking exons were amplified by PCR of donor plasmids pACD2 or pACD3 using the 5′primer pACD-T3 (5′-GGAGTCTAGAAATTAACCCTCACTAAAGGGGAATTGTGAGCG-3′), which appends a T3 promoter (underlined), and the 3′ primer LtrB+744a (5′-CTCCTCTAGAATCCGCTGTATCATCTAATATTCCTTTTG-3′). The PCR products were extracted with phenol-CIA (phenol/chloroform/isoamyl alcohol, 25:24:1 by volume), ethanol precipitated, and used as template for in vitro transcription with phage T3 RNA polymerase (Megascript T3 Kit; Ambion, Austin). After phenol-CIA extraction and ethanol precipitation, the precursor RNA containing the intron and flanking exons was self-spliced in reaction medium containing 1.25 M NH₄Cl, 50 mM MgCl₂, 50 mM Tris-HCl, pH 7.5 for 3 h at 37° C. 100 nM of the splicing products was then incubated with 200 nM of purified LtrA protein (00) in 10 ml of 450 mM NaCl, 5 mM MgCl₂, 40 mM Tris-HCl, pH 7.5 for 1 h at 30° C. Reconstituted RNPs were pelleted by ultracentrifugation in Beckman rotor 50.2Ti at 145000×g for 16 h at 4° C. and resuspended in 50 μl of 10 mM Tris-HCl, 1 nM DTTd, pH 7.5.

Xenopus oocyte preparation. Stage VI oocytes were manually peeled from the follicle cell layer in isolation medium (108 mM NaCl, 2 mM KCl, 1 MM EDTA, 1 mM HEPES, pH 7.5), treated with 0.05% collagenase (Sigma-Aldrich) for 10 min, and rinsed in incubation medium [Barth's solution (88 mM NaCl, 1 mM KCl, 0.82 mM MgSO₄, 2.4 mM NaHCO₃, 0.33 mM Ca(No₃)₂, 0.91 mM CaCl₂, 10 mM N-2-hydroxyethylpiperazine-N′-ethanesulphonic acid, pH 7.4) supplemented with penicillin (10,000 units/I), streptomycin (10 mg/I), gentamycin (50 mg/I), and theophylline (90 mg/I). Oocytes were kept in incubation medium at 16° C. for at least 30 min before injection.

Oocyte nuclei injection. Oocyte nuclear injection was done in incubation medium using a pressure system (Picospritzer III, Parker Hannifin, City and State) with 20 psi output. The injection volume was calibrated to 15-20 nl for each needle. A micromanipulator (MN-151, Narishige, City and State) was used to manipulate injection needles.

Plasmid targeting in Xenopus oocytes. 20 nl plasmid DNA (0.5 μg/μl) supplemented with MgCl₂ and dNTPs, as indicated for individual experiments, and 20 nl RNPs (1 μg/μl) were injected separately into Xenopus oocyte nuclei using different needles to avoid mixing before injection. For each set of experimental conditions, 10-20 oocytes were injected and pooled. The injected oocytes were rinsed twice in the incubation medium, incubated at 25-37° C. for different times, and stored at −80° C. For time course experiments, the oocytes were quick frozen in dry ice.

Total nucleic acids were isolated by incubating the oocytes in lysis buffer [20 mM Tris-HCl pH 8.0, 5 mM EDTA, pH 8.0, 400 mM NaCl, 1% SDS (w/v), 400 μg/ml proteinase K] for 1 h at 55° C., and then extracted twice with phenol-CIA. Nucleic acids were precipitated with isopropanol and dissolved in 20-50 μl of water. Two μl of total nucleic acid was electroporated into E. coli HMS 174 (DE3; F⁻, hsdR, reca, rif), and cells were plated at different dilutions on LB containing either amplicillin or ampicillin plus tetracycline. Colonies were counted after overnight incubation at 37° C., and the mobility frequency was calculated as ratio of (Amp^{R}+Tet^{R})/AmP^{R} colonies.

To detect trace amount of the homing product, total extracted nucleic acid was PCR-amplified using primers LtrB+456s (5′-TCTTGCAAGGGTACGGAGTAC) and pBRR+468a (5′-CCTTCTTAAAGTTAAACAAAATTATTTCTAGAGT), to amplify 3′ junction of the insertion, e.g., from position 456 of the inserted L1.LtrB intron, to 468-nt downstream of the insertion site in the recipient plasmid. The PCR products were purified in a 1% agarose gel and sequenced.

Results

Plasmid targeting assay in Xenopus oocyte nucleus. In this assay, an L1.LtrB intron with a phage T7 promoter inserted near its 3′ end inserts into the L1.LtrB target site (the ligated E1-E2 sequence of the ltrB gene) cloned upstream of a promoterless tet^{r} gene in an Amp^{R} donor plasmid (See FIG. 6A). Insertion of the intron containing the T7 promoter activates the tet^{r} gene, and intron-integration events are scored by the ratio of (Tet^{R}+Amp^{R})/Amp^{R} resistant colonies.

The protocol used for group II intron targeting reactions in Xenopus oocyte nuclei is shown in FIG. 6B. In initial experiments, the recipient plasmid pBRR3-LtrB (10 ng; 1.7×10⁹ molecules), which contains the wild-type ltrB target site, was injected into the nucleus in ten Xenopus oocytes nuclei, followed within one min by wild-type L1.LtrB-ΔORF RNPs (20 ng; 3.9×10¹⁰ molecules). In this and other experiments, the DNA target and RNPs were injected separately to avoid prior mixing. After the injection, the oocytes were pooled and incubated at 30° C. for 30 min, then nucleic acids were extracted, and electroporated into E. coli HMS174(DE3), which encodes an IPTG-inducible T7 RNA polymerase, to determine what proportion of the plasmids were Tet^{R}.

When the recipient plasmid DNA and intron RNP were injected without further additions, no Tet^{R} colonies were obtained. However, when 1 M MgCl₂ was added to recipient plasmid DNA before injection, RNP activity was increased dramatically, resulting in insertion frequencies of 9.8%. Sequencing of plasmids isolated from 14 independent transformants confirmed that the intron had integrated precisely at the target site (not shown), and controls showed that heat-inactivation of RNP particles at 90° C. for 3 min eliminated detection of mobility product either by PCR or Tet colony assay.

In early experiments, there was considerable variation in intron integration efficiency between different batches of oocytes (insertion frequences 0.002-7%). We found that addition of 17-20 mM dNTPs to the DNA/MgCl₂ injection mixture increased the insertion frequencies in suboptimal oocytes to the same 3-7% range found for good oocytes or even higher up to 20%. dNTP concentrations higher than 20 mM did not further increase the insertion frequencies. In subsequent experiments, 17 mM dNTPs were added routinely.

Mg²⁺ optimum. FIG. 7A shows a plot of the intron integration frequency as a function of injected MgCl₂ concentration. These and other experiment showed that the optimal Mg²⁺ concentration was ˜500 mM per 20 nl plasmid DNA. Assuming that the oocyte has a volume of 1 μl and that all of the injected Mg²⁺ remains in free form, then the injected Mg²⁺ would raise the total Mg²⁺ concentration by 10 mM. In other experiments, we found that 500 mM CaCl₂ or MnCl₂ could not substitute for MgCl₂, and that MgSO₄ or MgOAc gave only about 60% of the stimulation found for MgCl₂ (not shown). The order of injection of DNA and RNPs made no difference, and the Mg²⁺ could be injected separately from the DNA. However, there was no activity when RNPs were injected with 10-500 mM MgCl₂ (not shown).

In studies with bovine capillary endothelial cells, it was found that addition of 65 mM MgCl₂ to the culture medium results in chromosome unfolding (Bojanowski, K., Ingber, D. E. (1998) Ionic control of chromosome architecture in living and permeabilized cells. Exp. Cell. Res. 244, 286-94). While this may also occur in Xenopus oocyte nuclei and could be important for chromosome integration, we found that addition of 10-500 mM Mg²⁺ to the Xenopus oocyte incubation medium resulted in only a small increase in insertion frequency (from 0 to 4×10⁻⁴).

Temperature optimum. FIG. 7B shows a plot of the intron-integration frequency as a function of incubation temperature. For 30 min incubation in the temperature range of 16-37 ° C., higher temperature resulted in higher frequency, although 37 ° C. was deleterious to oocyte itself. With longer incubation, for 60-120 min, 25 and 30 ° C. resulted in high mobility, up to 20%, and had less visible effect on oocyte health (not shown). Therefore, 25 or 30 ° C. was used for further experiments.

Time courses. In time course experiments, after DNA and RNP injections, each group of oocytes were incubated at 25 or 30° C. for specified time and then quick frozen on dry ice, and product extracted and analyzed by Tet^{R} colony assay. The results are shown in FIGS. 7C and D. No mobility was detected when oocytes were frozen immediately after injections. Mobility product began to appear after 10 min and reached 8.0% and 9.4% at 120 min. Further incubation up to overnight did not increase the mobility efficiency.

To investigate the nature of the mobility products synthesized in Xenopus oocyte nuclei, nucleic acids extracted following the in vivo reaction were treated with Kpn I or Mlu I restriction enzyme prior to electroporation into E. coli and Tet^{R} colony assay. (FIG. 7C). KpnI and MluI each cuts at a single site with the inserted intron, when the recognition site part has been converted to double-strand DNA, but not efficiently when the substrate was single stranded, or DNA-RNA hybrid, which are the intermediate products of the intron homing reaction.

For 10 min sample, KpnI treatment did not change the mobility efficiency (0.006% to 0.006%). However, 20 min sample dramatically reduced TetR forming efficiency with KpnI treatment from 0.4% to 0.05%. As incubation time increases, the sensitivity to KpnI treatment tends to increase, suggesting progressive completion of the homing reaction into double-strand DNA. Treatment with MluI showed exactly the same trend.

Modified Group II Intron RNP Particles Specific to Xenopus Genes. L1.LtrB group II intron RNP particles modified to target Xenopus genes were prepared. Computer program designed oligonucleotides were used to alter intron specificity, and modified introns were cloned by PCR mediated method as described before (Perutka et al. 2004). Three introns targeted to Xenopus TX1 transposons, 5S RNA genes were cloned into vector pACD3 and targeted RNPs were reconstituted. The activity of the RNP in oocyte nuclei were confirmed by plasmid assay, using recipient pBRR3 plasmids with corresponding target sequence. Modified introns showed specific activities to its corresponding target sequences in oocyte nuclei.

RNPs targeted to three different target sequences in Xenopus genes, transposon Tx1 and 5s ribosomal DNA, were reconstituted in vitro as described, and injected into Xenopus oocyte nuclei, along with plasmid DNA containing corresponding target sequence, and appropriate amount of MgCl₂ and dNTP. Reacted DNA was extracted and assayed for Tetracycline resistance in E. coli.

| Xenopus target genes on plasmid | Mobility (%) |
|---|---|
| Tx1 2786 | 0.88 |
| Tx1 3772 | 0.01 |
| 5s DNA | 0.04 |

### Example 2

### RNP Injection into Zebrafish Embryo

In vitro fertilization of embryos was performed according to protocols described in The Zebrafish Book (Westerfield, M. (1989) The zebrafish book; A guide for the laboratory use of zebrafish (Brachydanio rerio). University of Oregon Press, Eugene, Oreg.). Approximately 0.5 mL of water was added to the sperm/egg mixture to allow fertilization to begin. Fertilized embryos were allowed to develop for approximately 15 minutes before injection.

The injection procedure was based on methods described in Zebrafish: A Practical Approach (Nusslein-Volhard, C. and Dahm, R. (2002) Zebrafish: A Practical Approach. Oxford. Universit. Press, Oxford). Borosilicate capillaries were pulled using a needle puller to prepare microinjection needles. The tip of the needle was cut to produce an open-ended point with a bore of approximately 10-15 μm. A needle was back-loaded with 2 μL of solution containing the RNP and phenol red tracer dye. A separate needle was back-loaded with target DNA solution and phenol red tracer dye. The needles were installed in a pressurized capillary holder connected to a Parker Picospritzer. Fertilized embryos were washed in 1× Steinberg reagent (69.0 mM NaCl, 680.0 μM KCl, 208 μM CaCl₂, 1.7 mM MgSO₄, 4.6 mM HEPES@ pH 7.6) and transferred from the 35 min petri dish to an injection plate. The injection plate consisted of an agarose slant that was designed to hold the embryos in place during injection. The embryos were covered with 1× Steinberg reagent. Injections were carried out by alternately injecting approximately 1-10 nL of RNP solution followed by 1-10 nL of DNA solution. The injection was targeted to the cytoplasm of the one-celled embryo. After 10 embryos were injected with both RNP and DNA, they were transferred to a fresh dish of 1× Steinberg reagent. The embryos were washed 6 times in fresh 1× Steinberg reagent and incubated at 30° C. or 37° C. for 30 minutes. After incubation, the embryos were immediately subjected to DNA extraction (see above). Extracted DNA was used for PCR detection of intron targeting product and for transformation of E. coli HMS174 (DE3) cells as described for Xenopus experiments. The PCR products were separated by gel electrophoresis using a 1% agarose gel. Appropriately sized products were band isolated and sequenced. Intron-integration events were scored by the ratio of (Tet^{R}+Amp^{R})/Amp^{R} resistant colonies following transformation of E. coli cells with the extracted DNA.

Results

Plasmid Targeting Assay in Danio rerio

Plasmid targeting assay with RNP injection, similar to the Xenopus experiment (see above) was performed using zebrafish embryos. In vitro fertilized, one-cell stage embryos were injected with target plasmid DNA, followed by separate RNP injection. Total DNA was extracted and analyzed by PCR and E. coli Tet^{R} assay. As in the Xenopus experiments, PCR products of expected sequence suggested RNP is active in Danio rerio embryo. Unlike in Xenopus, RNP activity in Danio rerio did not require addition of MgCl₂ for mobility to occur at levels detectable by PCR. However, addition of MgCl₂ did increase targeting efficiencies. Injection of plasmid DNA with MgCl₂ of increasing concentrations up to 0.150 M resulted in increased targeting efficiencies, as determined by the Tet^{R} assay, from 0.00042% targeting efficiency with 0.0 M MgCl₂ to 0.176% targeting efficiency at 0.150 M MgCl₂ similar to Xenopus.

### Example 3

### RNP Injection into Drosophila melanogaster Embryos

Mating cages were set up containing several male and female white 1118 flies. Female flies deposited fertilized embryos in the agar plates. The embryos were extracted from the agar and washed with sterile, distilled water. The embryos were manually dechorionated and placed on a slide to be transferred to a dessication chamber for approximately 5 minutes. Following dessication, the embryos were covered with oil. The fertilized embryos remained on the slide for injection.

The injection procedure was based on methods previously described, however the injection was manually controlled using a 50 mL syringe. Borosilicate capillaries were pulled using a needle puller to prepare microinjection needles. The tip of the needle was cut to produce an open-ended point with a bore of approximately 7-10 μm. A needle was back-loaded with 2 μL of solution containing the RNP. A separate needle was back-loaded with target DNA solution. The needles were connected to a capillary tubing that was attached to a 50 mL syringe. Injection of the embryos was performed by squeezing the syringe plunger while observing the expulsion of injection fluid. Injections were carried out by alternately injecting approximately 1-10 nL of RNP solution followed by 1-10 nL of DNA solution. The injection was targeted to the cytoplasm at the posterior end of the syncitial embryo. After approximately 40 embryos were injected with both RNP and DNA, they were incubated at 25° C. for 1 hour. Following incubation, the embryos were immediately subjected to DNA extraction (see above). Extracted DNA was used for PCR detection of intron targeting product and for transformation of E. coli HMS174 (DE3) cells as described for Xenopus experiments. The PCR products were separated by gel electrophoresis using a 1% agarose gel. Appropriately sized products were band isolated and sequenced. Intron-integration events were scored based on the ratio of (Tet^{R}+Amp^{R})/Amp^{R} resistant colonies following transformation of E. coli cells with the extracted DNA.

Results

Plasmid Targeting Assay in Drosophila melanogaster

Plasmid targeting assay with RNP injection, similar to the Xenopus and zebrafish experiments (see above) were performed using Drosophila embryos. Fertile flies (strain W1118) were allowed to mate and deposit fertilized embryos in agar plates made with apple juice. Embryos were extracted from the plates and washed with sterile, distilled water. Following manual dechorionation, the embryos were injected with RNP and the plasmid DNA containing the target site, as previously described. Total DNA was extracted and analyzed by PCR and E. coli Tet^{R} assay. As in the Xenopus and zebrafish experiments, PCR products of expected sequence suggested that the RNP is active in Droshophila melanogaster embryos. Similar to zebrafish embryos, RNP activity in Drosophila did not require the addition of MgCl₂ for mobility to occur at levels detectable by PCR. However, addition of MgCl₂ did increase targeting efficiencies, as determined by the Tet^{R} assay. Injection of plasmid DNA with MgCl₂ of increasing concentrations up to 0.100 M resulted in increased targeting efficiencies from 0.004% targeting efficiency with 0.0 M MgCl₂ to 0.05% targeting efficiency at 0.100 M MgCl₂ similar to Xenopus and zebrafish.

### Example 4

### Targeting DNA Substrates in Cultured Cells

Chromosomal Targeting in Tissue Culture by Electroporation of RNPs.

RNPs with GFP fused to the N-terminus of the LtrA protein. Among the conditions tested, (230 v, 100 μF) and (230 v, 250 μF) worked best on 0.4 ml of cells plus 10 μg of RNP. Twenty-four hours post electroporation, the majority of the surviving cells were transfected, judged with fluorescence microscopy (FIG. 8a). Flow cytometry showed a weak, but uniformly shifted peak of fluorescence intensity compared with untransfected cells (FIG. 8b). Immunofluorescence using anti-LtrA antibody showed similar signal pattern as GFP but stronger because the secondary antibody was conjugated with FITC, which has the same spectrum as GFP. RNPs were in speckles in the cells, some in the cytoplasm and some in the nucleus.

We then electroporated RNP preparations into 293 cells using the optimum condition (230 v, 250 μF) to target the rDNA. Cells were electroporated with or without RNP and treated with or without sodium butyrate (NaBu) before and after electroporated. Chromosomal DNA was prepared three days after electroporation, and nested PCRs were preformed to detect intron insertion. A PCR product of expected size for 3′ insertion junction was obtained from the DNA from cells not treated with NaBu (Chen & Pikaard, 1997) but electroporated with RNP. (FIG. 9A) The PCR product was confirmed as bearing correct intron/rDNA junction by sequencing (FIG. 9B). These results demonstrate that intron RNP can insert and reverse transcribe the 3′ end of the intron sequence.

### Methods

RNP Preparation

RNP was purified as described for LtrA protein purification Saldanha, R., et al., (Saldanha, R., Chen, B., Wank, H., Matsuura, M., Edwards, J., and Lambowitz, A. M. (1999) RNA and protein catalysis in group II intron splicing and mobility reactions using purified components. Biochemistry 38, 9069-9083) with the following modifications. The intron and LtrA sequences were cloned in expression vector, pImp-1p, so that both the intron RNA and the LtrA are under the control of T7 promoter. The C-terminus of LtrA is fused to the N-terminus of a chitin binding domain with an intein cleavage site, expressed downstream of the intron. The RNP construct was transformed to E. Coli BL21 (DE3), and a single colony was used to start an overnight culture. The next day, cells were subcultured at 1:50 and induced when OD₅₉₅ equals 0.6 with 0.5 mM of IPTG for 3 h at 25° C. Cells were collected and lysed in column buffer (500 mM NaCl, 25 mM Tris-HCl, pH 8.0, 0.1 mM EDTA) with 10 μg/ml of lysozyme by three cycles of freezing and thawing between −80° C. and 25° C. and brief sonication, and the lysate was loaded onto a chitin column for affinity purification. In order to minimize nuclease exposure, the flow rate was set at ≧1 mL/min. The loaded column was first washed with 100 mL of column buffer containing 750 mM of NaCl, then with 500 mM of NaCl. After overnight cleavage with 30 mM DTT, RNPs were eluted with two bed-volume of column buffer (500 mM NaCl) and ultracentrifuged overnight at 50,000 rpm and 4° C. The pellet was washed in ice-cold water and then resuspended in ice-cold 10 mM Tris-HCl, pH 8.0, and 1 mM DTT.

Electroporation of RNP

Cells were grown to about 80% confluence, trypsinized, neutralized with growth medium, and washed with ice-cold PBS at least 4 times. The cells were then resuspended in an appropriate volume of PBS to achieve a density of 5×10⁶/ml. Ten microgram of RNP was mixed with 400 μl of cells and incubated on ice for less than 2 min. Electroporations were carried out at 230 v and various capacitance values. After electroporation, to minimize the degradation of the RNPs by nucleases in the medium, cuvettes were left on ice for 10-15 min before 1 ml of growth medium was added to the cuvettes and cells were aliquoted to two 60-mm dishes. Fluorescence microscopy, flow cytometry, and immunofluorescence were performed 24 h after electroporation.

### Example 5

### Inserting an Exogenous Polynucleotide into an Endogenous DNA Substrate Using a Group II Intron RNP Particle and a DNA Construct

Preparation of Pickup fragment for homologous recombination. To construct pickup fragment (PUF) plasmid, a series of PCR amplification was performed. The first PCR amplified 700 bp upstream of pBRR3-ltrB intron insertion site (IIS), using primers pBRR-700BamS (5′-CGGCggatccTTCTTCGGGGCGAAAACTCTCAAGG-3′) and PUFNcoIT7A 5′-GAATTAAAAATGATATGCCCTATAGTGAGTCGTATTAccatggGTTATGGATGTGTT CAC-3′, using pBRR3-ltrB as template. The former primer introduced a BamHI site (in lower case) and the latter primer introduced a NcoI (in lower case) restriction site and a phage T7 promoter sequence (underlined).

The second PCR similarly amplified the 700bp downstream of IIS using PUFNcoIT7S (5′-GTGAACACATCCATAACccatggTAATACGACTCACTATAGGGCATATCATTTTTAA TTC-3′) and pBRR+70OBamA (5′-CGGCggatccCTGGGCGGCGGCCAAAGCGGTCGGA-3′). The PUFNcoIT7S sequence is exact reverse complement of PUFNcoIT7A, providing the second PCR fragment a homologous sequence to the first PCR fragment at the ends.

The third PCR using primers pBRR+700BamS and pBRR+700BamA, and mixture of purified products of the first and second PCR as template created continuous 1406 bp fragment, which contains 700 bp upstream, 700 bp downstream each of pBRR3-ltrB intron insertion site, with a NcoI restriction site and a phage T7 promoter sequence inserted at the intron insertion site.

The PCR fragment was cloned into pUC19 vector using the BamHI sites introduced by the primers at both ends. The correctness of insertion sequence was confirmed by sequencing. Finally, a 3967 bp NcoI fragment of bacteriophage lambda DNA was inserted at the NcoI site. The resulted PUF plasmid was digested by BamHI to cleave PUF out of the vector backbone, phenol-CIA extracted and ethanol precipitated before used for injections. The mixture of vector backbone fragment and PUF was injected without isolation of the fragment.

To test the efficacy of this approach in Xenopus oocytes, we used the experimental design shown in FIG. 10. In this assay, the closed-circular recipient plasmid pBRR3-LtrB (5 ng), containing the L1.LtrB intron-insertion site (IS), was co-injected with a 5.4-kb linear pickup fragment (PUF; 5 ng). The latter consists of a 4-kb DNA segment (3967-bp phage λ DNA NcoI fragment; positions 23902-27868) with a T7 promoter flanked by two 700-bp segments homologous to either side of the intron-insertion site in the recipient plasmid. Then L1.LtrB RNPs, without an internal T7 promoter, were injected into the same oocyte nuclei. Introduction of a double-strand break at the L1.LtrB target target site by the intron RNPs is expected to stimulate homologous recombination resulting in insertion of the PUF fragment with the T7 promoter in front of the tet^{R} gene. The insertion event was again assayed by the ability of extracted DNA to produce Tet^{R} colonies in E. coli. The injected oocytes were incubated at 30° C. for 1 h, followed by incubation at room temperature for 16 h. Then total nucleic acid was extracted and tested for its ability to give Tet^{R} colonies in E. coli.

The results are summarized in Table 2. The frequency of Tet^{R} colonies in the presence of RNPs was 4.8%, compared to less than 0.05% (zero Tet colony) in the absence of RNPs, at least 100-fold stimulation. RNP which was heat inactivated at 95° C. for 3 minutes did not show any activity. No Tet^{R} colony was observed in the reaction without PUF or without target plasmid. Addition of 500 mM MgCl₂ was essential to the reaction, but dNTP had no significant effect. Sequencing of plasmid DNA isolated from eight Tet^{R} colonies confirmed that the expected insertion of the PUF fragment.

What is claimed is:

1. A method of targeting a DNA substrate in a eukaryotic host cell, comprising;
a) introducing a purified group II intron RNP particle comprising a wild-type or modified group II intron RNA associated with a modified or wild-type group II intron encoded protein into the cell;
wherein the modified group II RNA comprises hybridizing sequences that allow the modified group II intron RNA to hybridize with recognition sequences in the endogenous polynucleotide; and
wherein the purified RNP particle is introduced into the eukaryotic cell by microinjection or electroporation; and
b) maintaining the host cell under conditions that allow the group II intron RNP particle to catalyze cleavage of the intracellular DNA substrate at a target site and to introduce a group II intron encoding the wild-type or modified group II intron RNA into the target site.

2. The method of claim 1 further comprising introducing magnesium ions into the host cell.

3. The method of claim 2, wherein magnesium ions are introduced into the host cell by introducing a solution comprising 100 mM or greater magnesium ions into the host cell.

4. The method of claim 3, wherein a solution comprising 100 mM to 1 M of a magnesium salt is injected into the host cell.

5. The method of any one of claims 1-2, wherein the group II intron RNA comprises an exogenous polynucleotide which is located in domain IV of a modified group II intron RNA.

6. The method of any one of claims 1-2, wherein the group II intron encoded protein is attached to a nuclear localization signal.

7. The method of claim 5, wherein the exogenous polynucleotide encodes a protein other than the group II intron encoded protein.

8. The method of any one of claims 1-2, wherein the host cell is an embryonic stem cell, a fertilized or unfertilized oocyte, a sperm or a zygote.

9. The method of any one of claims 1-2, wherein the host cell is a tissue culture cell.

10. The method of any one of claims 1-9, wherein the group II intron is inserted into a coding sequence, gene segment, or regulatory element.

11. The method of claim 5, wherein the exogenous polynucleotide encodes a therapeutic product selected from the group consisting of enzymes, cytokines, hormones, antigens, antibodies, clotting factors, regulatory proteins, ribozymes, transcription proteins, receptors, and anti-sense nucleic acid molecules.

12. A method of disrupting expression of a targeted gene in a eukaryotic cell, comprising a) introducing a purified RNP particle comprising a modified or wild-type group II intron RNA associated with a modified or wild-type group II intron encoded protein into the cell,
wherein the group II intron RNA comprises sequences that are complementary to sequences in or near the targeted gene,
wherein the purified RNP particle is introduced into the cell by microinjection or electroporation; and
b) maintaining the eukaryotic cell under conditions that allow the RNP particle to cause formation of a single stranded or double-stranded break at a site in or near the targeted gene.

13. The method of claim 12, wherein the group II intron RNA is modified to base pair with a specific sequence in or near the targeted gene.

14. The method of claim 12, wherein the host cell is an embryonic stem cell, a sperm, a plant cell, a fertilized or unfertilized oocyte, or a zygote.

15. The method of claim 12, wherein the host cell is a tissue culture cell.

16. A method of inserting an exogenous polynucleotide into a target site in a DNA substrate in a eukaryotic cell, comprising:
introducing into the eukaryotic cell a wild-type or modified group II intron ribonucleoprotein (RNP) particle configured to introduce a single or double stranded break at a target site in a DNA substrate in the cell and a DNA construct comprising an exogenous polynucleotide flanked by sequences that are homologous to sequences that flank the target site in the endogenous DNA substrate, and
maintaining the eukaryotic cell under conditions that permit the modified RNP particle to introduce the single or double-stranded break at the target site in the DNA substrate and that permit insertion of the DNA construct into the target site.

17. The method of claim 16, wherein the group II intron RNP particles are introduced into the cells by electroporation or microinjection.

18. The method of claim 16, wherein the group II intron RNP particles are introduced into the cell by expressing one or more polynucleotides that encode the modified group II intron RNA and the group II intron encoded protein and that have been introduced into the cell.

19. The method of any one of claims 16-18, wherein the DNA substrate is an endogenous gene or chromosomal locus, and wherein insertion of the exogenous polynucleotide into the target site results in deletion of a coding sequence, gene segment, or regulatory element; alteration of a coding sequence, gene segment, or regulatory element; insertion of a new coding sequence, gene segment, or regulatory element; creation of a conditional allele; or replacement of a coding sequence or gene segment from one species with an homologous or orthologous coding sequence from the same or a different species.

20. The method of claim 16, wherein the eukaryotic cell is an animal cell or a plant cell.

21. A method of targeting a DNA substrate in a eukaryotic host cell, comprising;
a) introducing a purified group II intron RNP particle comprising a wild-type or modified group II intron RNA associated with a modified or wild-type group II intron encoded protein into the cell;
wherein the modified group II RNA comprises hybridizing sequences that allow the modified group II intron RNA to hybridize with recognition sequences in the endogenous polynucleotide;
b) introducing magnesium ions into the cell, and
c) maintaining the host cell under conditions that allow the group II intron RNP particle to catalyze cleavage of the intracellular DNA substrate at a target site and to introduce a group II intron encoding the wild-type or modified group II intron RNA into the target site.
